(19) United States
(12) Patent Application Publication
(10) Pub. No.: US 2016/0264932 A1
(43) Pub. Date: Sep. 15, 2016

(54) METHOD FOR PREPARING CULTURE MEDIUM FOR CULTURING PLANT TISSUE, METHOD FOR CULTURING PLANT TISSUE, STERILIZING AGENT, MICROBICIDAL AGENT, AND CULTURE MEDIUM COMPOSITION FOR CULTURING PLANT TISSUE

(71) Applicant: Youichi MIZUTA, Kyoto-shi (JP)

(72) Inventor: Youichi MIZUTA, Kyoto-shi (JP)

(21) Appl. No.: 14/778,420

(22) PCT Filed: Sep. 20, 2013
(86) PCT No.: PCT/JP2013/075495

§ 371 Date: Sep. 18, 2015

(51) Int. Cl. C12N 5/00

(52) U.S. Cl. CPC C12N 5/0025

(57) ABSTRACT

A culture medium for culturing a plant tissue includes: boiling a culture medium; adding a first sterilizing agent composed of a plurality of kinds of powdery agents to the culture medium; adding a second sterilizing agent composed of a single kind of agent to the culture medium; dispensing the culture medium into a culture vessel; cooling the dispensed culture medium; making the cooled culture medium and a plant tissue wet with a plurality of kinds of liquid agents; and introducing the plant tissue in the culture medium. By this invention, a method for preparing a culture medium for culturing a plant tissue and a method for inoculating a plant tissue can be provided in which the sterilizing or microbicidal treatment can be easily performed and yet the plant tissue can be grown to approximately the same extent as in the case of using an autoclave and a clean bench.

### TECHNICAL FIELD

The present invention relates to a method for preparing a culture medium used for culturing a plant tissue and a method for culturing a plant tissue, as well as a sterilizing agent, a microbicidal agent, and a culture medium composition for culturing a plant tissue.

### BACKGROUND ART

Normally, in the culturing of a plant tissue, either a culture medium in the form of a gel or solid prepared by adding a gelling agent (e.g. agarose or gellan gum) to a culture solution, or a liquid culture medium which contains no gelling agent is used. The culture medium is contained in a culture vessel, and in this state, the medium is subjected to a sterilizing treatment, after which the plant tissue is placed in the culture medium under a sterile environment (this task is hereinafter called the “inoculation”) and the culturing of this plant tissue is performed in an incubation room. Such a procedure is necessary since, if germs are allowed to enter the culture vessel, the germs will multiply due to sugar and other nutrients in the culture medium and consequently impede the growth of the plant tissue.

Normally, the sterilization of the culture medium and the culture vessel is performed with a high pressure steam sterilizer (autoclave), while the inoculation of the plant tissue is performed in a sterile room (clean bench). Accordingly, after the sterilization, the culture medium or the like must be removed from the autoclave and transferred to the sterile room. This task is cumbersome since it requires a great amount of care to avoid entry of the germs. Another problem is that the capacity of the autoclave or sterile room limits the amount of plant tissue that can be cultured, so that it is impossible to simultaneously culture a large amount of plant tissue.

Accordingly, a method by which a plant tissue can be easily cultured, even outdoors, without using a high pressure steam sterilizer and sterile room has been researched and developed.

For example, Non Patent Literature 1 (which is an academic paper put forth by the present inventor and other authors) discloses a simple method for culturing a plant tissue using a plurality of kinds of agents including a chlorine microbicide. This method includes the steps of heating a culture medium to boiling temperature to dissolve the medium, adding agents a plurality of times to the dissolved medium to sterilize the medium, immersing the culture vessel and the plant body in a liquid containing a microbicidal agent to kill bacteria, and culturing the plant body under a normal environment. According to a report in Non Patent Literature 1, the thereby obtained sterilizing or microbicidal effect is approximately comparable to those obtained by using a high pressure steam sterilizer and sterile room.

### CITATION LIST

### Non Patent Literature

Non Patent Literature 1: Yoichi Mizuta, Kiyoaki Miyasaka, Yusuke Muraishi, and Motoaki Doi, “Ooto-kureebu To Kuriin Benchi Wo Mochiinai Kan-i Soshiki Baiyou Ni Okeru Biseibutsu Osenritsu No Teigen (Reduction of Microbial Contamination Rate in a Simple Tissue Culturing Method without Using Autoclave and Clean Bench)”, Horticultural Research (Japan), Vol. 9, Additional Volume 2, September, 2010, p. 298

### SUMMARY OF INVENTION

### Technical Problem

However, the plurality of kinds of agents used in the previously described method cannot be previously mixed. Therefore, it is necessary to separately weigh each of those agents immediately before adding them to the culture medium or immersing the culture vessel and the plant body in a liquid. Clearly, such a task is complex and troublesome.

The problem to be solved by the preset invention is to provide a method for preparing a culture medium for culturing a plant tissue and a method for culturing a plant tissue in which the sterilizing or microbicidal treatment can be easily performed and yet the plant tissue can be grown to approximately the same extent as in the case of using an autoclave and a clean bench, as well as a sterilizing agent for a culture medium for culturing a plant tissue, a microbicidal agent for a plant body, and a culture medium composition for culturing a plant tissue.

### Solution to Problem

A method for preparing a culture medium for culturing a plant tissue according to the present invention developed for solving the previously described problem includes the successive processes of:

boiling a culture medium;

adding a first sterilizing agent composed of a plurality of kinds of powdery agents to the culture medium at a point in time in the boiling process;

adding a second sterilizing agent composed of a single kind of agent to the culture medium at the end of the boiling process, and dispensing the culture medium into a culture vessel; and

cooling the dispensed culture medium.

The present invention is characterized in that the first sterilizing agent to be added at a point in time in the process of boiling the culture medium is composed of a plurality of kinds of powdery agents, and the second sterilizing agent to be added at the end of the boiling process is composed of a single agent. The mixing of the different powdery agents does not alter the nature of those agents. Therefore, the first sterilizing agent can be previously prepared.

The “point in time in the boiling process” does not need to be earlier than the “end of the boiling process”; they may be at the same point in time. If the “point in time in the boiling process” coincides with the “end of the boiling process”, the first and second sterilizing agents may be previously mixed. In this case, the method for preparing a culture medium for culturing a plant tissue according to the present invention will be substantially identical to the method including the successive processes of:

boiling a culture medium;

adding a sterilizing agent composed of a plurality of kinds of powdery agents to the culture medium at a point in time in the boiling process, and subsequently dispensing the culture medium into a culture vessel; and

cooling the dispensed culture medium.

The agents composing the first sterilizing agent should preferably contain one or more substances selected from the group of sucrose fatty acid esters, nisin, natamycin, ε-polylysine, protamines, citrate, and hypochlorite, among which sucrose fatty acid esters and nisin are particularly preferable. Glycerin fatty acid esters may also be contained in addition to or in place of the sucrose fatty acid esters.

The agent composing the second sterilizing agent should preferably contain one or more substances selected from the group of captan, oxolinic acid, and hypochlorite. For the first and second sterilizing agents, agents or substances which are officially designated as food additives or agricultural chemicals should preferably be used.

A method for culturing a plant tissue according to the present invention developed for solving the previously described problem includes the processes of:

immersing a culture vessel containing a solid or semisolid culture medium in a first microbicidal composition solution containing a chlorine microbicide for a predetermined period of time;

immersing a plant tissue in a second microbicidal composition solution for a predetermined period of time; and

inoculating the plant tissue removed from the second microbicidal composition solution in the culture medium contained in the culture vessel removed from the first microbicidal composition solution, and culturing the plant tissue in the culture medium.

As the culture medium contained in the culture vessel to be immersed in the first microbicidal composition solution, the culture medium prepared by the previously described method can be preferably used.

The first microbicidal composition solution should preferably contain an agent containing sucrose fatty acid esters, in addition to the chlorine microbicide. The second microbicidal composition solution should preferably contain agents which respectively contain sucrose fatty acid esters, captan, oxolinic acid and natamycin. In this case, the agents containing the sucrose fatty acid esters and natamycin should preferably be products officially designated as food additives, while both the agent containing captan and the agent containing oxolinic acid should preferably be products officially designated as agricultural chemicals.

Furthermore, the first microbicidal composition solution may also contain CMIT (e.g. ZonenC or Kathon) in addition to the previously mentioned substances.

A sterilizing agent for a culture medium for culturing a plant tissue according to the present invention is characterized by being composed of a powdery agent containing at least sucrose fatty acid esters and nisin.

A culture medium composition for culturing a plant tissue according to the present invention is characterized by containing the previously mentioned sterilizing agent and a powdery culture-medium component.

A microbicidal agent used for culturing a plant tissue according to the present invention is an agent to be used for a microbicidal treatment of a plant body or culture vessel, characterized by being composed of a plurality of kinds of powdery agents mixed together.

### Advantageous Effects of the Invention

In the method for preparing a culture medium for culturing a plant tissue according to the present invention, since the first sterilizing agent and the second sterilizing agent can be previously prepared, it is possible to easily kill bacteria in a culture vessel and culture medium without having to perform a cumbersome task. Furthermore, by using the obtained culture medium, the plant tissue can be grown to approximately the same extent as in the case of using an autoclave and a clean bench.

In the method for culturing a plant tissue according to the present invention, the microbicidal treatment can be easily performed by merely immersing the culture medium and the plant tissue in the first and second microbicidal composition solutions, respectively. The plant tissue cultured by this method can grow to approximately the same extent as in the case of using an autoclave and a clean bench.

The sterilizing agent for a culture medium for culturing a plant tissue, the microbicidal agent for a plant body, and the culture medium composition for a plant tissue according to the present invention previously contain necessary components for the sterilization of a culture medium or the microbicidal treatment of a plant body. Therefore, by using this agent or composition, the sterilization of the culture medium and the microbicidal treatment of the plant body can be easily performed.

### BRIEF DESCRIPTION OF DRAWINGS

FIG. 1 illustrates a procedure for sterilizing a solid culture medium (two-time dosing mode).

FIG. 2 illustrates a procedure for sterilizing a solid culture medium (one-time dosing mode).

FIG. 3 illustrates a procedure for sterilizing a solid culture medium (medium-component mixture mode).

FIG. 4 illustrates a culturing procedure using a solid culture medium.

FIG. 5 illustrates a culturing procedure using a liquid culture medium.

FIG. 6 shows an experimental result showing the influence of the agent concentration on the growth of plants.

FIG. 7 shows an experimental result showing the influence of the combination and concentrations of agents on the sterilization of a culture medium.

FIG. 8 shows an experimental result showing the influence of the combination and concentrations of agents in the culture medium on the microbial recontamination after solidification.

FIG. 9 shows an experimental result showing the influence of the concentrations and combination of agents in the inoculating process on the sterilizing effect.

FIG. 10A shows an experimental result showing the occurrence rate of microbial contamination.

FIG. 10B shows an experimental result showing the occurrence rate of microbial contamination.

FIG. 11 shows the result of a comparative experiment on the growth of explants.

FIG. 12 shows the result of a comparative experiment showing how the growth of explants changes depending on the method used for sterilizing a liquid culture medium.

FIG. 13 shows the result of an experiment on a change in the occurrence rate of the microbial contamination in the liquid culture.

FIG. 14 shows the result of an experiment on the occurrence rate of the microbial contamination of a liquid culture medium after sterilization.

FIG. 15 shows the result of an experiment on present examples and comparative examples.

### DESCRIPTION OF EMBODIMENTS

The method for culturing a plant tissue and the method for preparing a culture medium for culturing a plant tissue according to the present invention are hereinafter described with reference to specific examples. Initially described are a method for sterilizing a culture medium and a method for the microbicidal treatment of a culture vessel. The following tables 1-5 respectively show the kinds of agents, culture vessels, culture-medium compositions, inoculated bacteria, and explants, all of which were used in the sterilization examples, etc.

TABLE 1
Used Agents

| | Brevity | Product | Product | Main Component | | | |
|---|---|---|---|---|---|---|---|
| | Code | Name | State | Name | Content | | Manufacturer |
| Food | SE-L | RYOTO | Viscous | Sucrose | 280 | g/kg | Mitsubishi- |
| Additives | | Sugar Ester | liquid | monolaurate | | | Kagaku Foods |
| | | LWA-1570 | | | | | |
| | SE-P | RYOTO | Powder | Sucrose | 800 | g/kg | Mitsubishi- |
| | | Sugar Ester | | monopalmitate | | | Kagaku Foods |
| | | P-1670 | | | | | |
| | Nata | Natamycin | Powder | Natamycin | 950 | g/kg | A and Z Food |
| | | | | | | | Additives Co., |
| | | | | | | | Ltd. |
| | Nisin | Nisaplin | Powder | Nisin A | 25 | g/kg | Danisco |
| | Imp | Impact N | Powder | Salmine | 60 | g/kg | Asama |
| | | | | | | | Chemical Co., |
| | | | | | | | Ltd. |
| | Ly | Egg-white | Powder | Egg-white | 950 | g/kg | A and Z Food |
| | | lysozyme | | lysozyme | | | Additives Co., |
| | | | | | | | Ltd. |
| Agricultural | Cl | Chemichlon G | Grain | Available chlorine | 700 | g/kg | Nippon Soda |
| Chemicals | | | | in hypochlorous | | | Co., Ltd. |
| | | | | acid | | | |
| | Cap | Orthocide- | Powder | Captan | 800 | g/kg | Sankei |
| | | 80 | | | | | Chemical Co., |
| | | | | | | | Ltd. |
| | Ox | Wettable | Powder | Oxolinic acid | 200 | g/kg | Sumika Agro |
| | | powder | | | | | Manufacturing |
| | | STARNER | | | | | Co., Ltd. |
| Environmental | Zc | ZonenC | Water- | 5-chloro-2- | 133.6 | g/L | Chemicrea |
| Disinfectants | | | soluble | methylmethyl-4- | | | Inc. |
| | | | liquid | isothiazolinone | | | |
| | PHMB | Supermill88 | Water- | polyhexamethylene | 40 | g/L | Epro |
| | | | soluble | biguanide | | | Corporation |
| | | | liquid | | | | |

TABLE 2

| Culture Vessel | Specification |
|---|---|
| Test tube | A glass tube measuring 23 mm in inner diameter and 150 mm in length, |
| | with a polypropylene (PP) molten plug. Capacity for culture medium, |
| | 25 mL |
| Plastic case | A PP case measuring 155 mm in width, 250 mm in length and 200 mm |
| (for solid culture medium) | in depth, with a 20-L polyethylene bag fitted therein and sealed by tying |
| | its top. Capacity for culture medium, 1 L. |
| Plastic case | A PP case measuring 155 mm in width, 250 mm in length and 200 mm |
| (for liquid culture medium) | in depth, with a 45-L polyethylene bag fitted therein and sealed by |
| | folding it in half and tying its top. Capacity for culture medium, 5 L. |

TABLE 3

| | Culture Medium | |
|---|---|---|
| | Composition | Remarks |
| Common | Raw water: tap water | |
| Features | Granulated sugar, 15 g/L | |
| Gelling Agent | Gellan gum, 2 g/L | Only used for solid culture media. |
| Salt | Otsuka-A | Otsuka House 1 Gou + Otsuka House 2 Gou |
| Composition | MS | Murashige & Skoog's inorganic salt and EDTA |
| | Knop | Knop's salt, Murashige and Skoog's micro nutrients, and |
| | | EDTA |
| Additive | +Y&P | Bact Yeast Extract (2.5 g/L) and Bact Tripton (5 g/L) |
| Organic | +oatmeal | 7.5 g/L |
| Substance | +mashed potato | 7.5 g/L |
| | +coconut milk | 75 g/L |
| | +coconut water | 75 g/L |
| | +potato | 75 g/L, peeled, and ground with a mixer to fluid state |
| | +banana | 75 g/L, peeled, and ground with a mixer to fluid state |
| | +beef liver | 75 g/L, peeled, and ground with a mixer to fluid state |

TABLE 4

| Inoculated | |
|---|---|
| Bacteria | |
| (Brevity | |
| Code) | |
| B5 | A bacterial suspension of B5 strain, presumed to be |
| | Bacillus subtilis, containing 10¹⁰ cfu · mL⁻¹ of spores |
| Gst | A bacterial suspension of Geobacillus stereothermophyros |
| | ATTC7953 containing 10⁸ cfu · mL⁻¹ of spores |
| S3 | A bacterial suspension of S3 strain, presumed to be Serratia |
| | sp., containing 10¹² cfu · mL⁻¹ of viable bacteria |
| An | A conidial powder of a filamentous fungus, presumed to be |
| | Aspergillus niger complex. The conidial powder contains |
| | 3 × 10⁶ CFU/mg of conidia. |

TABLE 5

| Name | | | | |
|---|---|---|---|---|
| (Breed) | Abbreviated Name | Condition | Weight Per Piece | |
| Potato | One node of potato | One node of plant cultured in vitro, | 0.008-0.015 | g/explant |
| “May | (without leaves) | without leaf blades | | |
| Queen” | One node of potato | One node of plant cultured in vitro, with | 0.015-0.03 | g/explant |
| | (with leaves) | leaf blades | | |
| | Potato shoot | The entire plant cultured in vitro, with | 0.15-0.3 | g/explant |
| | | the underground part removed | | |
| Chrysanthemum | One node of | One node of plant cultured in vitro, | 0.02-0.04 | g/explant |
| “Piato” | chrysanthemum | without leaf blades | | |
| | (without leaves) | | | |
| | One node of | One node of plant cultured in vitro, with | 0.1-0.2 | g/explant |
| | chrysanthemum | leaf blades | | |
| | (with leaves) | | | |
| | Chrysanthemum | The entire plant cultured in vitro, with | 2-4 | g/explant |
| | shoot | the underground part removed | | |
| Carnation | One node carnation | One node of carnation cultured in vitro | 0.15-0.3 | g/explant |
| “Francesco” | | | | |
| Saintpaulia | Saintpaulia | One leaf blade of plant cultured in vitro | 0.01-0.025 | g/explant |
| “Polar Star” | | | | |
| Taro | Taro | A few nodes including terminal bud of | 0.1-0.3 | g/explant |
| “Eguimo” | | plant cultured in vitro | | |
| Northern | Northern | A plant cultured in vitro | 0.02-0.04 | g/explant |
| Maidenhair | maidenhair fern | | | |
| Fern | prothallium | | | |

[Sterilization of Solid Culture Medium]

### (1) Sterilization of Culture Medium in Solidifying Process

Solid culture media were sterilized by the following methods (1-1) through (1-3). A solid culture medium obtained by those methods is hereinafter called the “sterilized solid culture medium.”

(1-1) A method in which the culture medium is sterilized by separately adding two doses of sterilizing agents in the process of solidifying the culture medium. (This method is hereinafter called the “two-time dosing mode.” See FIG. 1.)

1. A culture medium is heated and boiled.

2. A first sterilizing agent (whose composition will be described later) is added to the dissolved culture medium.

3. The boiling state is maintained for three minutes.

4. After a second sterilizing agent (whose composition will be described later) is added, the culture medium is dispensed into culture vessels and cooled at room temperature to solidify it.

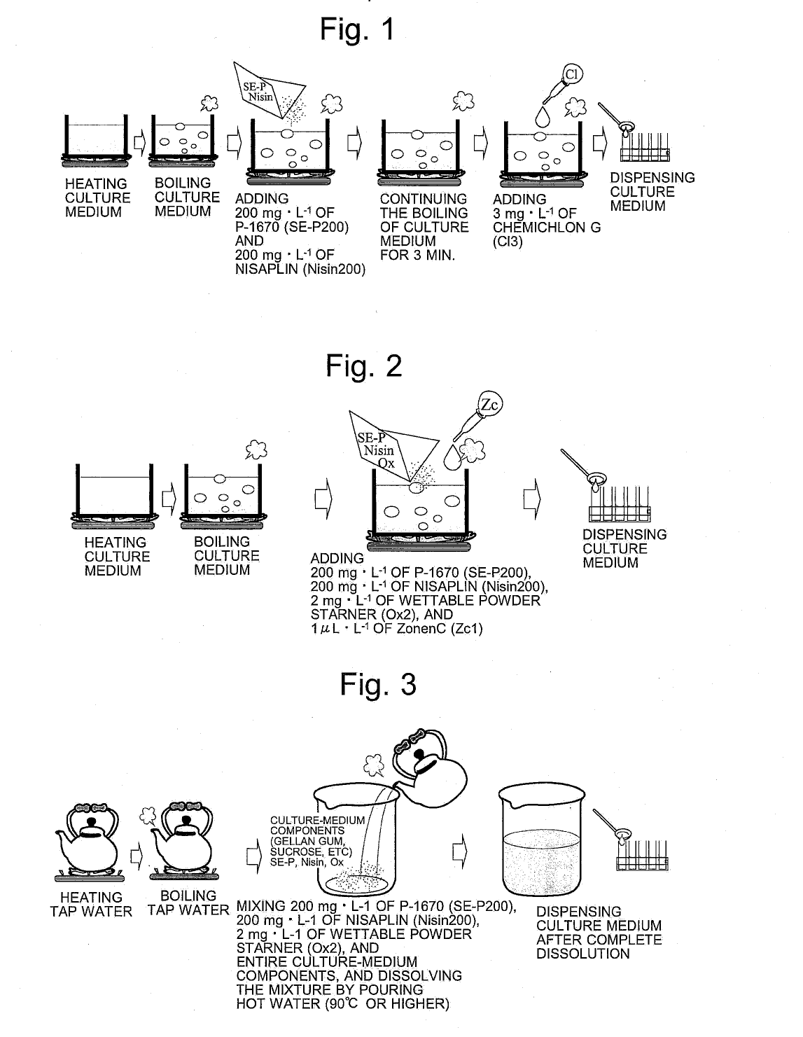

(1-2) A method in which the culture medium is sterilized by adding one dose of sterilizing agent in the process of solidifying the medium. (This method is hereinafter called the “one-time dosing mode.” See FIG. 2.)

1. A culture medium is heated and boiled.

2. After a first sterilizing agent (whose composition will be described later) is added to the dissolved culture medium, the medium is dispensed into culture vessels and cooled at room temperature.

(1-3) A method in which sterilizing agents are previously mixed in the culture-medium components. (This method is hereinafter called the “medium-component mixture mode.” See FIG. 3.)

1. An amount of tap water is heated and boiled.

2. A mixture of sterilizing agents and all the culture-medium components is dissolved by pouring hot water of 90° C. or higher onto that mixture.

3. After the culture medium is completely dissolved, the medium is dispensed into culture vessels and cooled at room temperature.

### [Method of Inoculating (Culturing) Explant]

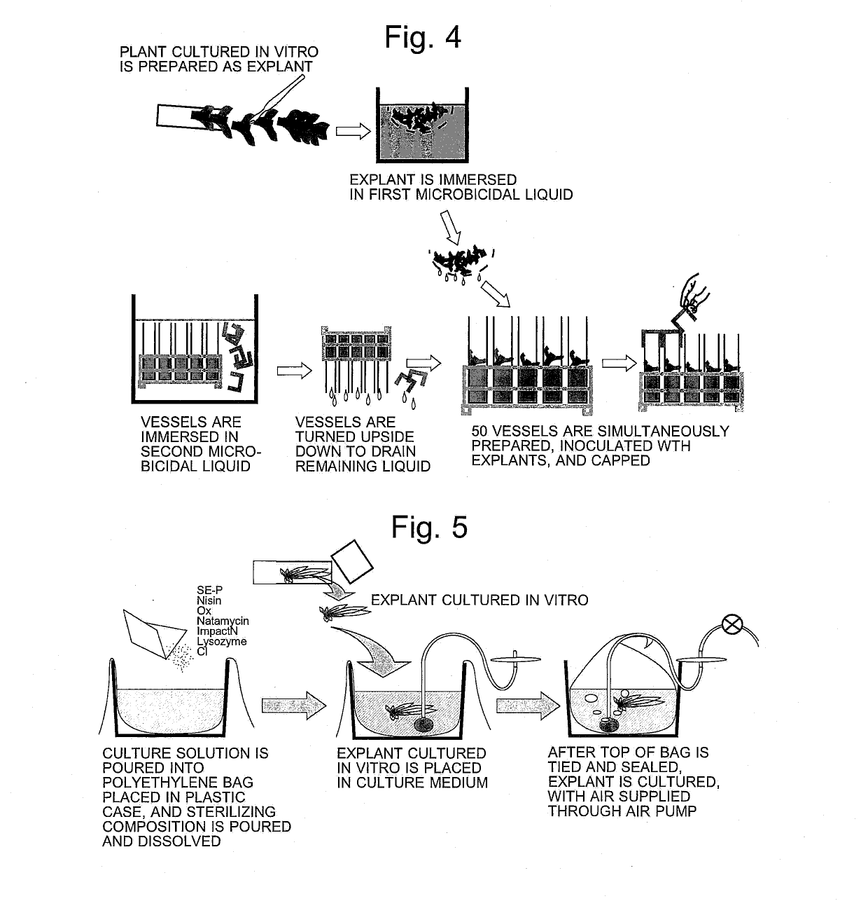

### (1) Culturing on Solid Culture Medium (See FIG. 4)

1. An explant is immersed in a first microbicidal liquid and is subsequently removed from the liquid and drained. The first microbicidal liquid is previously prepared by dissolving, in an amount of tap water, one or more agents selected from the following agents: 2000 mg/L of P-1670 (SE-P2000), 2000 mg/L of Nisaplin (Nisin2000), 500 mg/L of wettable powder STARNER(Ox500), 10000 mg/L of Natamycin (Nata10000), 10000 mg/L of Orthocide-80 (Cap1000), and 20000 mg/L of Impact N (Imp20000).

2. A sterilized solid culture medium contained in the culture vessels is immersed in a second microbicidal liquid, which contains one or both of the following agents: 2000 mg/L of P-1670 (SE-P2000) and 1400 mg/L of Chemichlon G (Cl1400).

3. The culture vessels are removed from the second microbicidal composition liquid and turned upside down to drain the remaining liquid.

4. The explant obtained in Step 1 is inoculated on the culture medium in the culture vessels obtained in Step 3, and is cultured in a standard laboratory.

### (2) Culturing in Liquid Culture Medium (See FIG. 5)

1. A polyethylene bag is placed in a plastic case, and a culture solution is poured into the polyethylene bag.

2. A microbicidal agent is added to and dissolved in the culture solution. The microbicidal agent is one or more of the following agents: 10 mg/L of P-1670 (SE-P10), 10 mg/L of Nisaplin (Nisin10), 2 mg/L of wettable powder STARNER (Ox2), 50 mg/L of Natamycin (Nata50), 50 mg/L of Orthocide-80 (Cap50), 100 mg/L of Impact N (Imp100), 100 mg/L of egg-white lysozyme (Ly100) and 7 mg/L of Chemichlon G (Cl7).

3. A germ-free explant is inoculated in the culture solution obtained in Step 2, and is cultured in a normal laboratory.

The specific results of the experiments are hereinafter described.

[Influence of Agent Concentration on Growth of Plant]

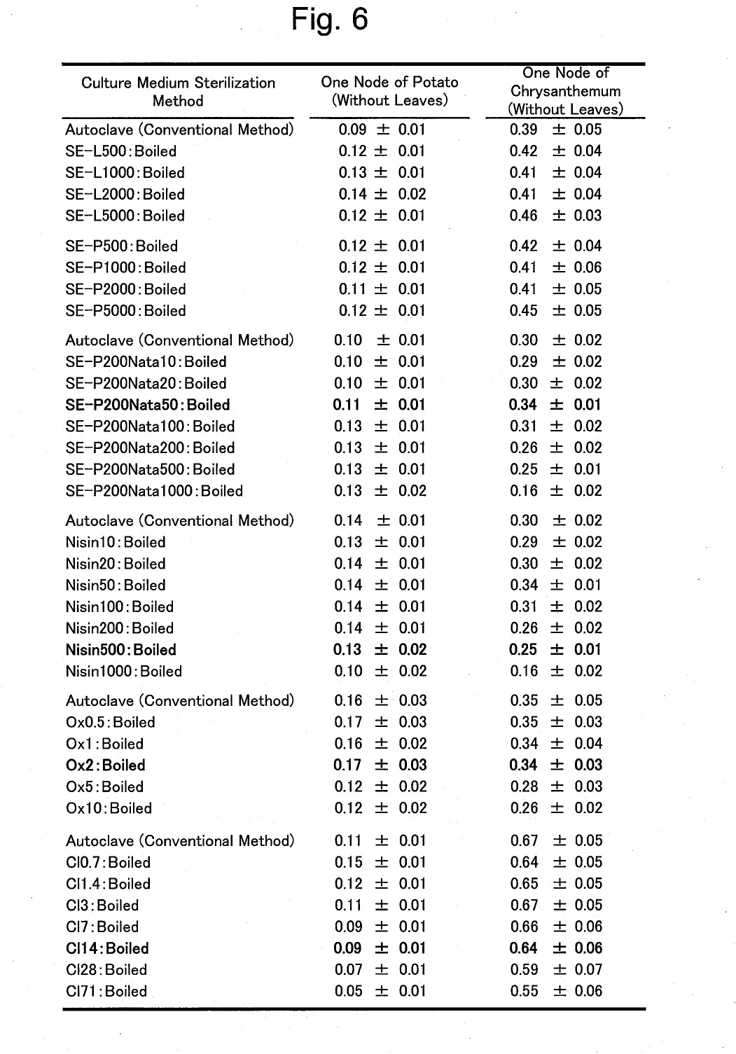

The influence of a change in the concentrations of the agents used for sterilizing the culture medium on the growth of plants was investigated. The result is shown in FIG. 6. The culture condition in this experiment was as follows:

Culture medium components: solid culture medium dosed with Otsuka-A

Culture period: 30 days

Number of plant bodies: 10

Numerical values: average±standard deviation

Sterilizing method: one-time dosing mode

Inoculating method (Culturing method): the method shown in FIG. 4 was used, with SE-P2000Cl140/SE-P2000Cl140 as the first/second microbicidal liquids, respectively.

In FIG. 6, the “Autoclave (Conventional Method)” indicates comparative examples, in which an explant which had undergone a microbicidal treatment in an autoclave was placed in a liquid culture medium and cultured in a clean bench.

As can be seen in FIG. 6, P1670 (SE-P) and LWA1570 (SE-L) did not negatively affect the growth of the plant when their concentration in the culture medium was 5 g/L or lower.

The natamycin agent (Nata) did not negatively affect growth when its concentration was 100 mg/L or lower. The reason why P1670 (SE-P) was added with the natamycin agent was because using only natamycin allows nonnegligible contamination by spore-forming bacteria.

Nisaplin (Nisin) did not negatively affect growth when its concentration was 500 mg/L or lower.

Wettable powder STARNER (Ox) did not negatively affect growth when its concentration was 2 mg/L or lower.

Chemichlon G (Cl) did not negatively affect growth when its concentration was 14 mg/L or lower. The concentration of the available chlorine in 14 mg/L of Chemichlon G is 10 mg/L.

Based on these results, the upper limits of the concentrations of the agents used in each sterilizing agent or microbicidal liquid were determined.

### [Influence of Concentrations and Combination of Agents on Sterilization of Culture Medium]

The sterilization effect on bacteria inoculated in the solid culture medium was investigated. The culture condition was as follows:

Culture period: 30 days

Culture condition: 40° C., in the dark

Number of test tubes used in the measurement: 50

Numerical values: microbial contamination rate (the percentage of the tubes in which microbial colonies were formed)

Inoculated amount: 1 mL per 1 L of the culture medium

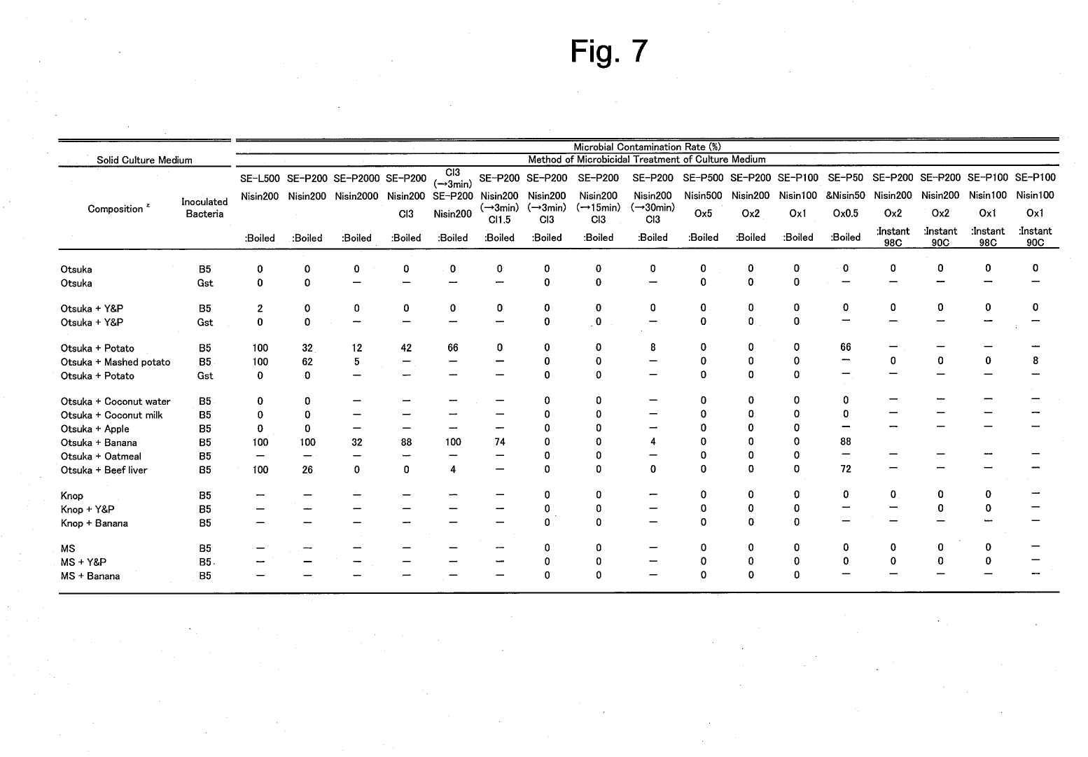

The result is shown in FIG. 7. A comparison between LWA1570 and P1670 in FIG. 7 demonstrates that P1670 produces a higher degree of sterilization effect. The following facts can also be realized: Some kinds of culture media cannot be sterilized even if the agent concentration is increased; Chemichlon G must not be simultaneously added with LWA1570 or P1670; and Chemichlon G must not be added before the addition of LWA1570 or P1670.

In particular, Chemichlon G should be added within 3-15 minutes from the addition of P1670 or Nisaplin. Though the procedure of preparing the culture medium becomes slightly complex, this method allows the agent to be composed of only food additives. This method is also advantageous in that there is no need to use an oxolinic acid agent (wettable powder STARNER) which has significantly adverse effects on plants. When P1670, Nisaplin and wettable powder STARNER are combined, all of these agents can be simultaneously added.

[Influence of Concentrations and Combination of Agents in Culture Medium on Microbial Recontamination after Solidification]

A sterilized culture medium (Otsuka-A solid culture medium) was placed in 50 test tubes held in a tube stand and kept intact on a standard laboratory table (at temperatures of 14-24° C.), and the temporal change in the degree of microbial colony formation was investigated.

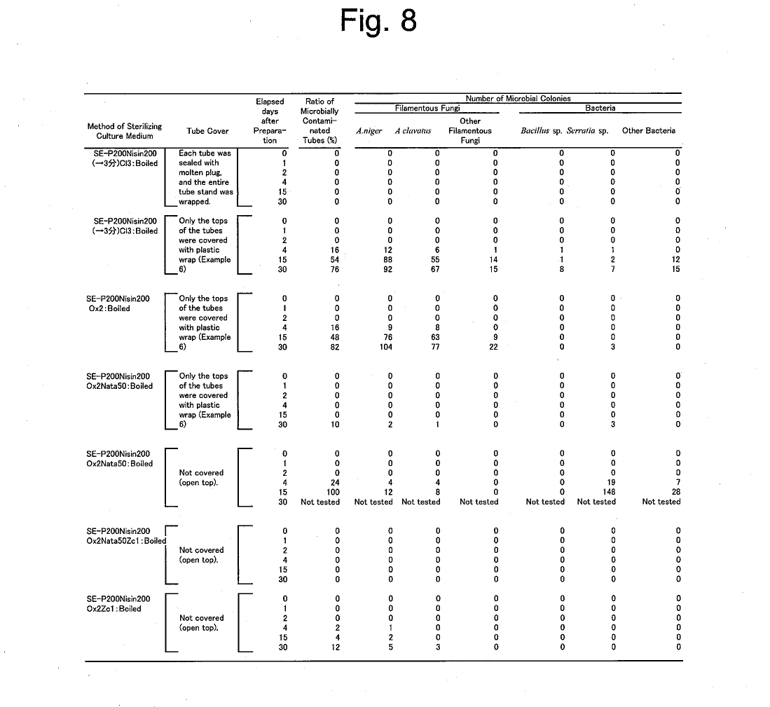

The result is shown in FIG. 8.

FIG. 8 demonstrates that no colony was formed on the culture medium sterilized with P1670, Nisaplin and Chemichlon G when the vessels were sufficiently sealed so as to prevent newly invasion of microbes (i.e. the sterilization is sufficient).

However, when the vessels were not sufficiently sealed, a considerable amount of microbes reentered and formed colonies.

When P1670, Nisaplin and wettable powder STARNER were used, newly invasion of bacteria was decreased. When Natamycin was also used in addition to those agents, newly invasion of filamentous fungi was also dramatically decreased, although open storage of the culture was not possible.

However, the open storage was made possible by further adding ZonenC. It was also found that, when Natamycin was not added, the open storage was impossible even when ZonenC was added.

[Influence of Concentrations and Combination of Agents in Inoculating Process on Sterilizing Effect]

An experiment was conducted to determine how the sterilizing effect after inoculation is related to the concentrations and combination of the agents used in the microbicidal liquid for explants and the microbicidal liquid for culture vessels. The experimental condition was as follows:

Solid culture medium: dosed with Otsuka-A

Sterilizing method: autoclave

Method of inoculation of bacteria: on culture medium surface [as for “B5” and “S3”, 50 μL per test tube of the bacterial suspension was dropped onto the surface of the culture medium; and as for “An”, 10 mg per test tube of either a conidial powder or a conidial powder diluted with a-starch to a predetermined dilution ratio was poured onto the culture medium]; or on explant [as for “B5” and “S3”, the explant was immersed in the bacterial suspension (whereby 10 μL per explant was attached); and as for “An”, the explant was coated with either a conidial powder or a conidial powder diluted with a-starch to a predetermined dilution ratio (whereby 5 mg per explant was attached)]

Explant: potato

Inoculation method: the method shown in FIG. 4

Culture period: 14 days

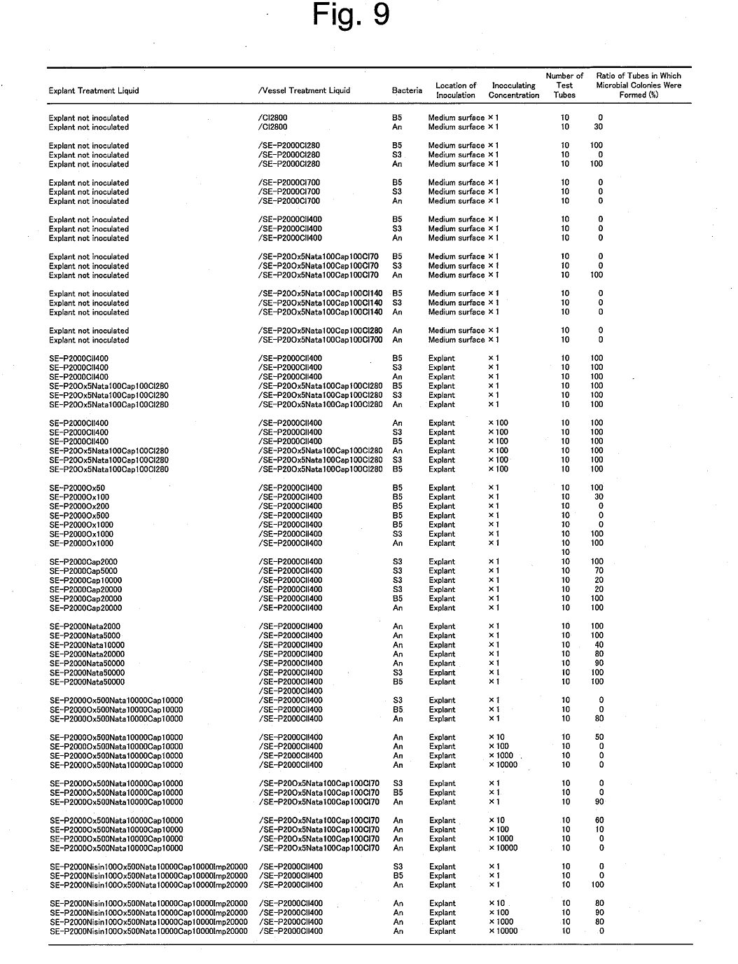

The result is shown in FIG. 9. From the result obtained by treating the culture vessel with Chemichlon G (Cl2800) without inoculating any explant, it has been determined that the formation of “An” colony could not be prevented even when the concentration of the available chlorine was increased to a high level of 2000 mg/L.

When P-1670 (SE-P) was added, the colony formation could be prevented by immersing the culture vessel in a liquid containing 700 mg/L or higher amounts of Chemichlon G (equivalent to 500 mg/L or higher amounts of available chlorine).

Furthermore, when Orthocide (Cap), wettable powder STARNER (Ox), Natamycin (Nata) or the like was added at low concentrations, the colony formation could be prevented even with a liquid whose Chemichlon-G concentration was decreased to 140 mg/L (equivalent to 100 mg/L of available chlorine).

When bacteria were inoculated on the explant, it was impossible prevent the colony formation by immersing the explant in the same microbicidal liquids as used in the case where no explant was inoculated.

The colony formation could not be prevented even when the inoculating concentration was decreased by a factor of one thousand.

“B5” could be sterilized by treating the explant with a microbicidal liquid containing 200 mg/L or higher amounts of wettable powder STARNER (Ox). However, this liquid could not prevent the formation of “An” and “S3” colonies. The formation of “S3” colony could be prevented to some extent by treating the explant with a microbicidal liquid which contained 10000 mg/L or higher amounts of Orthocide-80 (Cap). However, this liquid could not prevent the formation of “An” and “B5” colonies. The formation of “An” colony could be prevented to some extent by treating the explant with a microbicidal liquid which contained 10000 mg/L or higher amounts of Natamycin (Nata). However, this liquid could not prevent the formation of “S3” and “B5” colonies. When all of the three kinds of agents were added, the three kinds of bacteria could be prevented from forming colonies (for “An”, this effect could be obtained when its inoculating concentration was decreased by a factor of one hundred or more). Such a tendency barely changed even when the kind of microbicidal liquid for treating the culture vessel was altered.

Adding Nisaplin (Nisin) and Salmine lowered the microbicidal effect on “An”, rather than improving it.

[Occurrence Rate of Microbial Contamination]

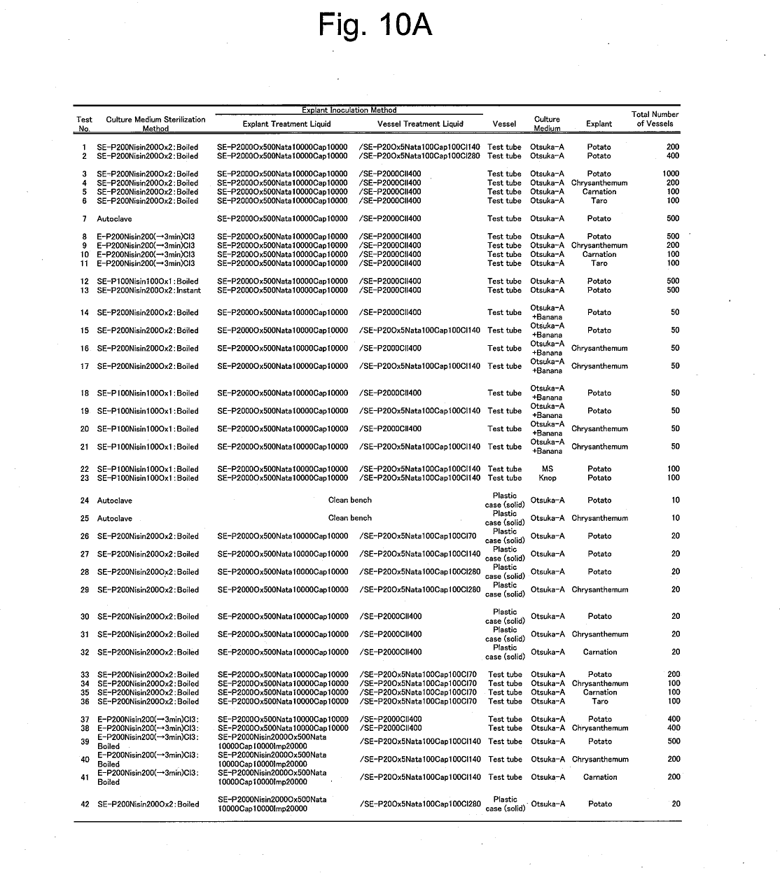

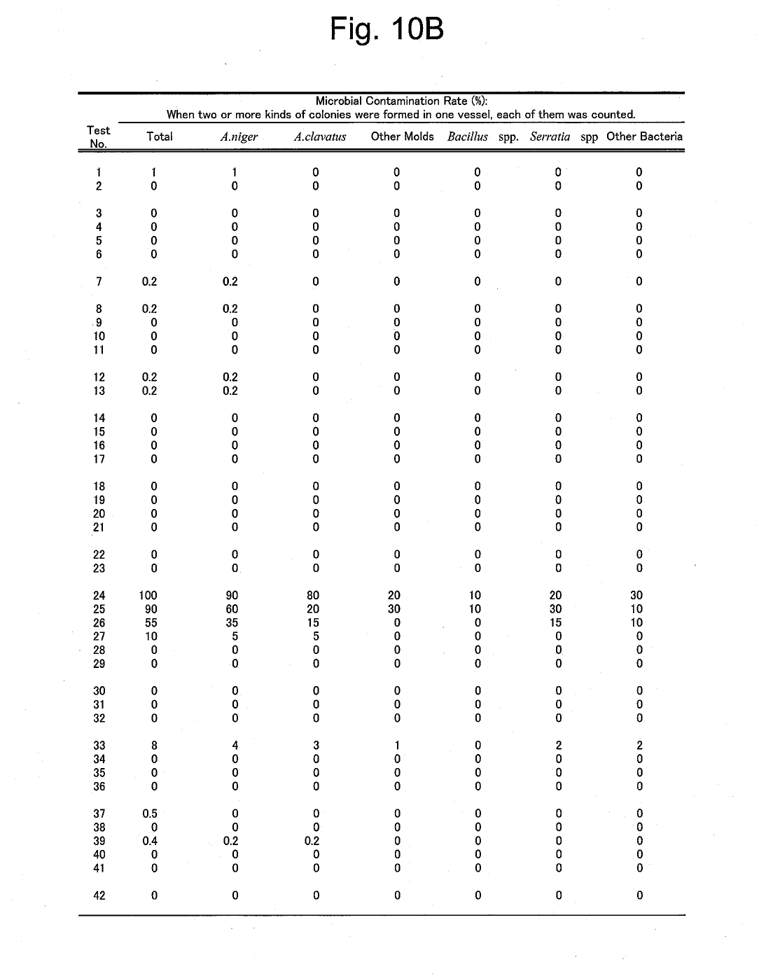

The occurrence rate of contamination by external microbes after inoculation was investigated for various combinations of the sterilizing method for culture media, treatment liquid for explants, and treatment liquid for culture vessels. The list in FIG. 10A shows the kinds of sterilizing composition, microbicidal liquid and other information on each experiment. FIG. 10B shows the occurrence rate of microbial contamination.

The information in these figures demonstrates that the microbial contamination can be prevented by appropriately determining the kinds of agents and their concentrations. In particular, the contamination of the potato, which is prone to being contaminated by microbes in the inoculating process, could be suppressed by increasing the agent concentrations.

### [Comparison of Growth of Explants]

Based on the previously described results, sterilizing compositions and microbicidal liquids were prepared by appropriately combining the agents with concentrations determined so as to avoid adverse effects on the growth of explants. Using those compositions and liquids, explants were actually cultured and their growths were compared. The culture condition was as follows:

Culture vessel: test tube

Culture medium: Otsuka-A, solid

Culture condition: cultured for 30 days, with one explant in each test tube

Number of measurements: 10 test tubes

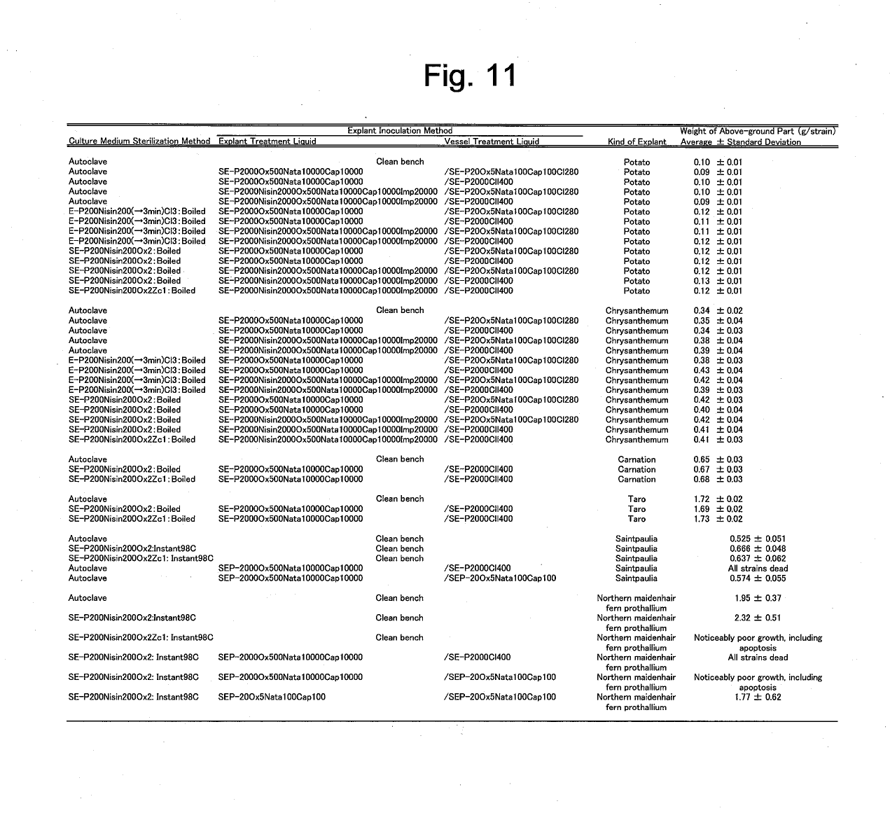

The result is shown in FIG. 11. As can be seen in FIG. 11, for most of the plants, whichever methods were used for the culturing and medium sterilization did not significantly affect their growth. However, Saintpaulia and northern maidenhair fern prothallium, both of which are sensitive to agents, could not be inoculated without a clean bench. It is most likely that, for such plants, not only the microbicidal liquid applied to the explant but also ZonenC contained in the culture medium is also harmful. It should be noted that Saintpaulia, northern maidenhair fern prothallium and the like could also be successfully cultured by omitting the addition of ZonenC to the culture medium or diluting the microbicidal liquid used in the explant-inoculating process.

[Comparison of Growth of Explants Due to Difference in Method of Sterilization of Liquid Culture Medium]

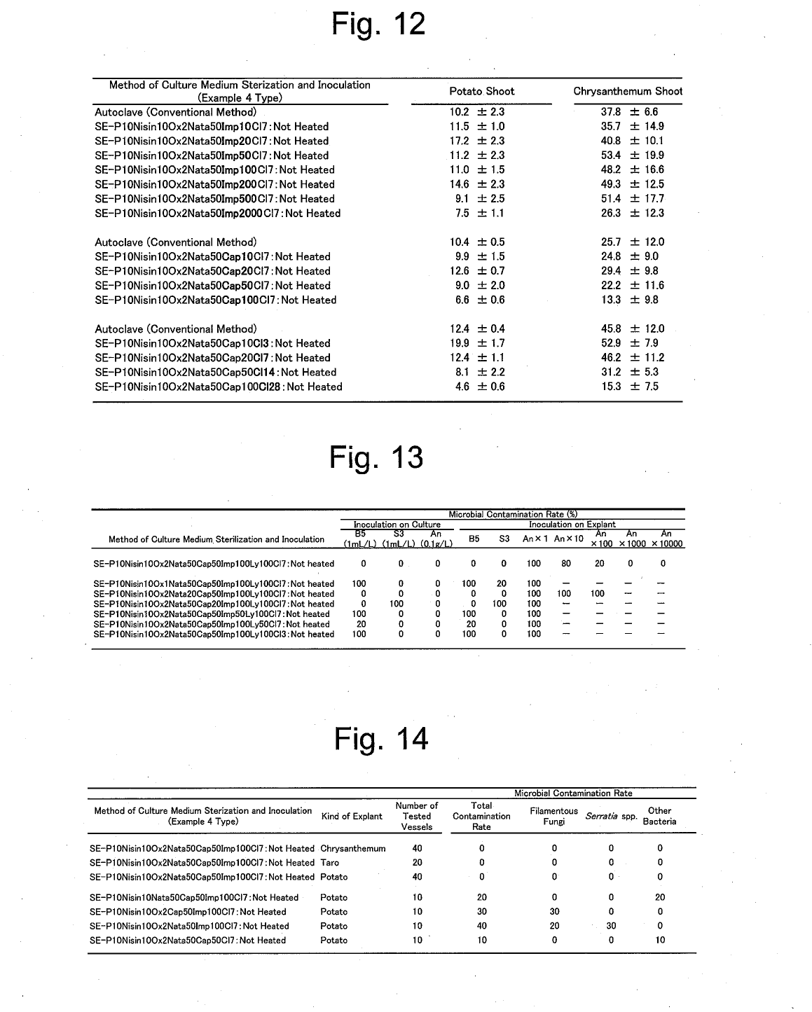

Concerning the liquid culture, an experiment was conducted to investigate a change in the growth of explants depending on the method of sterilizing the liquid culture medium. Explants were placed in a liquid culture medium dosed with Otsuka-A and cultured for 30 days. The result is shown in FIG. 12. From FIG. 12, the following facts have been established: Impact N does not adversely affect growth when its concentration is 500 mg/L or lower; Orthocide-80 does not adversely affect growth when its concentration is 50 mg/L or lower; and Chemichlon G, when its concentration is 7 mg/L or lower, can produce a sufficient sterilizing effect for achieving a growth which equals or even exceeds the growth achieved by the autoclaving method.

[Difference in Occurrence Rate of Microbial Contamination in Liquid Culture]

The microbial contamination rate was compared between the case of inoculating bacteria in the culture medium and the case of inoculating bacteria on the explant.

The inoculating concentration of the bacteria, the kind of plant body, etc. were as follows:

Culture medium: As for “B5” and “S3,” 1 mg/L was added to the culture medium. As for “An”, 0.1 g/L of its conidial powder was added.

Plant body: An explant (potato shoot) which had been immersed in a culture solution of “B5” or “S3” was inoculated, or an explant coated with diluted conidospores of “An” was inoculated.

Number of measurements: 5 bags, each sample

The result is shown in FIG. 13. As can be seen in FIG. 13, in the case of the liquid culture medium, similarly to the solid culture medium, the microbes inoculated in the culture medium showed a higher contamination rate than those inoculated on the explant. The microbial contamination rate by “An” was particularly high, which proves that this type of contamination is difficult to prevent.

[Occurrence Rate of Microbial Contamination of Liquid Culture Medium after Sterilization]

The contamination rate by external bacteria after sterilization of a liquid culture medium was investigated. In any cases, the result was obtained under the condition that the inoculation of explants was performed in a common room with no bacteria inoculated. The result is shown in FIG. 14. As can be seen in FIG. 14, adding the egg-white lysozyme was not necessary to prevent microbial contamination. The microbial contamination rate increased when any one of the following agents was omitted: SE-P, Nisin, Ox, Nata, Cap, Imp10 and Cl7.

Specific examples of the present invention and the comparative examples will be hereinafter described. The culture medium and culture vessel used for the culturing as well as other information are as follows:

### (1) Culture Medium

A culture medium dosed with Otsuka-A (not diluted)+ammonium sulfate (0.5 g/L), sucrose (15 g/L) and gellan gum (2 g/L)

Yeast extract (2.5 g/L), peptone (5 g/L) and other ingredients (75 g/L, crushed with a mixer) were added to the culture medium.

### (2) Culture Vessel

A test tube with an inner diameter of 23 mm, with a plastic molten plug

### (3) Amount of Culture Medium

30 mL, with one explant inoculated in each culture vessel

### (4) Culture Condition

“20° C.+Explant”: white fluorescent lamp 30 μmol/m²·s, 16-hour day length, day/night temperature=23° C./20° C.

“38° C.”: 38-40° C. in the dark

### (5) Culture Period

For approximately 30 days from the day after the culture medium was prepared

### (6) Explant

A piece (one or two nodes) of a cultured plant body of potato or chrysanthemum

### (7) Number of Cultured Plants

50 pieces (25 pieces of potato and 25 pieces of chrysanthemum)

### (8) Inoculated Thermoduric Spore and Its Density

Thermoduric spores of B5 strain (presumed to be Bacillus subtilis) were inoculated at a density of 1×10¹⁰ CFU per 1 L of culture medium.

The culturing was performed either in the one-time dosing mode or in two-time dosing mode.

### Example 1

The process steps in this example are as follows:

1. After the culture medium is heated and boiled, a first sterilizing agent composed of a mixture of SE-P (0.2 g/L) and nisin (0.2 g/L) is added to the dissolved culture medium. Both SE-P and nisin are powdery agents.

2. The boiling state is maintained for two minutes.

3. After Cl (3 mg/L) as a second sterilizing agent is added, the culture medium is dispensed into the culture vessels and cooled at room temperature to solidify it.

### Example 2

This example is the same as Example 1 except that the boiling is continued for five minutes. The process steps are as follows:

1. After the culture medium is boiled, a first sterilizing agent composed of a mixture of SE-P (0.2 g/L) and nisin (0.2 g/L) is added.

2. The boiling state is maintained for five minutes.

3. After Cl (3 mg/L) is added, the culture medium is dispensed into the culture vessels and cooled at room temperature.

### Example 3

This example is the same as Example 1 except that the boiling is continued for 10 minutes. The process steps are as follows:

1. After the culture medium is boiled, a first sterilizing agent composed of a mixture of SE-P (0.2 g/L), nisin (0.2 g/L) and A (5 mg/L) is added.

2. The boiling state is maintained for 10 minutes.

3. After Cl (2 mg/L) is added, the culture medium is dispensed into the culture vessels and cooled at room temperature.

### Example 4

This example is the same as Example 1 except that the boiling is continued for 30 minutes. The process steps are as follows:

1. After the culture medium is boiled, a first sterilizing agent composed of a mixture of SE-P (0.2 g/L) and nisin (0.2 g/L) is added.

2. The boiling state is maintained for 30 minutes.

3. After Cl (2 mg/L) is added, the culture medium is dispensed into the culture vessels and cooled at room temperature.

Comparative Examples are hereinafter described. The process steps in Comparative Examples are basically the same as those of Examples 1-4 except that each of the two kinds of agents was individually weighed and added after the culture medium is boiled as well as after the continued boiling is completed.

### Comparative Example 1

1. After the culture medium is heated and boiled, Cl (1.4 mg/L) and SE-L (0.5 g/L) are individually weighed and added.

2. The boiling state is maintained for three minutes.

3. After Cl (1.4 mg/L) and nisin (0.2 g/L) are individually weighed and added, the culture medium is dispensed into the culture vessels and cooled at room temperature.

### Comparative Example 2

This example is the same as Comparative Example 1 except that SE-L (which is one of the agents added to the culture medium after the culture medium is boiled) is changed to SE-P which also contains sucrose fatty acid esters. The process steps are as follows:

1. After the culture medium is heated and boiled, Cl (1.4 mg/L) and SE-P (0.2 g/L) are individually weighed and added.

2. The boiling state is maintained for three minutes.

3. After Cl (1.4 mg/L) and nisin (0.2 g/L) are individually weighed and added, the culture medium is dispensed into the culture vessels and cooled at room temperature.

### Comparative Example 3

In this example, all of the four kinds of agents are added after the culture medium is boiled, and the obtained culture medium is immediately dispensed into the culture vessels. The process steps are as follows:

1. After the culture medium is heated and boiled, Cl (3 mg/L), SE-P (0.2 g/L) and nisin (0.2 g/L) are individually weighed and added.

2, The culture medium is dispensed into the culture vessels and cooled at room temperature.

### Comparative Example 4

In this example, the entire amount of Cl is added earlier, while SE-P is added later. The process steps are as follows:

1. After the culture medium is heated and boiled, Cl (3 mg/L) is weighed and added.

2. The boiling state is maintained for five minutes.

3. After SE-P (0.2 mg/L) and nisin (0.2 g/L) are individually weighed and added, the culture medium is dispensed into the culture vessels and cooled at room temperature.

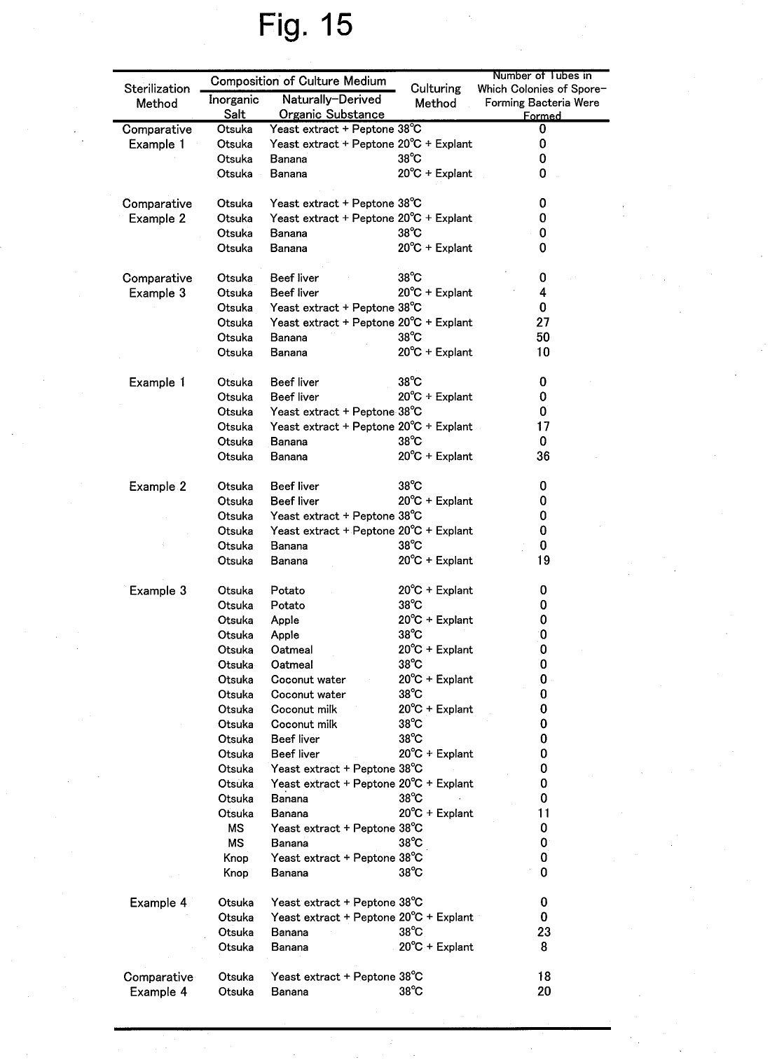

Using the culture media prepared by the methods of Examples 1-4 and Comparative Examples 1-4, the culturing was performed for 30 days by the previously described culturing method. The result is shown in FIG. 15.

In Comparative Examples 1 and 2 (which are conventional culture-medium sterilization methods developed by the present inventor), there was no test tube in which a colony of spore-forming bacteria was formed, which demonstrates that sufficient sterilizing effects can be obtained by those methods. However, the agents used in Comparative Examples 1 and 2 cannot be previously mixed. Specifically, SE-L used as the source of SE in Comparative Example 1 is a viscid liquid. Although nisin is a powdery agent, its nature may possibly alter if it is previously mixed, since Cl, which is to be simultaneously added, is a strong oxidizing solid. In Comparative Example 2, SE-P used as the source of SE is a powdery agent and cannot be previously mixed as well, since Cl, which is to be simultaneously added, is a strong oxidizing solid. Accordingly, it is necessary to perform the cumbersome task of individually weighing and adding the two kinds of agents in the boiling phase as well as after the continued boiling.

By contrast, Comparative Example 3 is an example in which all the agents are simultaneously added in order to decrease the amount of labor. However, in this example, colonies of spore-forming bacteria were formed in some of the culture media, which means that the sterilizing effect was lower than Comparative Examples 1 and 2.

On the other hand, in Examples 1-4, the two agents (SE-P and nisin) to be added in the boiling phase are powdery agents and can be previously mixed. Furthermore, there is only one kind of agent to be added after the continued boiling. Therefore, in terms of the number of times of weighing the agents and the adding task, Examples 1-4 are superior to Comparative Examples 1 and 2. As for the sterilizing effect, which was measured by the number of test tubes in which colonies of spore-forming bacteria were formed, Example 1 (with a boiling period of two minutes) was inferior to Comparative Examples 1 and 2, but was superior to Comparative Example 3. Examples 2, 3 and 4 with longer boiling periods of 5, 10 and 30 minutes, respectively, produced even higher sterilizing effects: colonies of spore-forming bacteria were formed only on the culture medium to which banana was added. In particular, in Examples 2 and 3, the colony formation ratio of the spore-forming bacteria on the banana-added culture medium was as low as 5×10⁻¹⁰. Thus, it has been proved that sufficient sterilizing effects can be obtained.

In any of the previous examples, food additives were used as the agents for sterilizing culture media. It is also possible to use agricultural chemicals. Furthermore, the following variations are also possible.

### <Variation 1>

1. After the culture medium is boiled, 0.25 mg/L of a mixed agent composed of P-1670, Nisaplin, natamycin, wettable powder STARNER (agricultural chemical) and dextrin mixed at a ratio by weight of 100:100:10:1:39 is added.

By this Variation 1, the amount of bacteria entering the culture medium after the cooling and solidifying step can be decreased, so that the culture vessel does not need to be sealed when it is stored.

### <Variation 2>

1. A mixture of 0.25 g/L of a mixed agent (composed of P-1670, Nisaplin, natamycin, wettable powder STARNER (agricultural chemical) and dextrin mixed at a ratio by weight of 100:100:10:1:39), 20 g/L of granulated sugar, 2 g/L of gellan gum, 1.5 g/L of Otsuka House 1 Gou and 1 g/L of Otsuka House 2 Gou is prepared. Subsequently, 1 L of boiled water is poured onto 25 g/L of the obtained mixture and thoroughly stirred to dissolve the mixture.

According to this Variation 2, a sterilized culture medium can be created by simply pouring hot water onto an amount of powder.

### <Variation 3>

This is a culture medium sterilization method suitable for performing the liquid culturing at room temperature. In this method, 0.25 g/L of a mixed agent (composed of P-1670, Nisaplin, natamycin, Impact N, Orthocide-80 and wettable powder STARNER (agricultural chemical) mixed at a ratio by weight of 4:4:20:51:20:1) and 7 mg/L of Chemichlon G are added to a culture medium having an approximately room temperature, and the culture medium is sealed.

By this method, a plant culture medium having a simple composition mainly containing typical salts, vitamins and plant growth regulators can be sufficiently sterilized if the degree of contamination by thermoduric spores is not higher than approximately 10000 CFU/L.

### <Variation 4>

This is a method for sterilizing a plant tissue (a plant piece or plant body), which uses agricultural chemicals and food additives. Specifically, a plant tissue is immersed in an aqueous solution containing P-1670 (1 g/L), natamycin (10 g/L), wettable powder STARNER (10 g/L) and Orthocide-80 (10 g/L). Subsequently, the plant tissue is introduced in a culture medium sterilized by the method of Example 5 or Variation 1, and the vessel is sealed. In the case of Example 5, the culture medium is immersed together with its vessel in a solution containing 1 g/L of P-1670 and 1.43 g/L of Chemichlon G (hypochlorite).

By this method, plants with almost any degree of external contamination can be sterilized.

### <Variation 5>

This is a method for sterilizing a plant tissue, which uses food additives. Specifically, a plant is immersed in an aqueous solution of P-1670 (1 g/L), natamycin (10 g/L) and Nisaplin (10 g/L). Subsequently, the plant is introduced in a culture medium sterilized by the method of Examples 5 or Variation 2, and the vessel is sealed. In the case of Example 5, the culture medium is immersed together with its vessel in a solution containing P-1670 (1 g/L) and Chemichlon G (1.43 g/L).

The degree of sterilization by this method is sufficiently high to perform the sterile culturing of an explant without using a clean bench.

1-13. (canceled)

14. A method for preparing a culture medium for culturing a plant tissue, comprising successive processes of:
boiling a culture medium;
adding a first sterilizing agent composed of a plurality of kinds of powdery agents to the culture medium at a point in time in the boiling process;
adding a second sterilizing agent composed of a single kind of agent to the culture medium at an end of the boiling process, and dispensing the culture medium into a culture vessel; and
cooling the dispensed culture medium.

15. The method for preparing a culture medium for culturing a plant tissue according to claim 14, wherein the agents composing the first sterilizing agent contains one or more substances selected from a group of sucrose fatty acid esters, nisin, natamycin, ε-polylysine, protamines, citrate, and hypochlorite.

16. The method for preparing a culture medium for culturing a plant tissue according to claim 14, wherein the agent composing the second sterilizing agent contains one or more substances selected from a group of captan, oxolinic acid, and hypochlorite.

17. A method for culturing a plant tissue, comprising processes of:
immersing a culture vessel containing a solid or semisolid culture medium in a first microbicidal composition solution containing a chlorine microbicide for a predetermined period of time;
immersing a plant tissue in a second microbicidal composition solution for a predetermined period of time; and
inoculating the plant tissue removed from the second microbicidal composition solution in the culture medium contained in the culture vessel removed from the first microbicidal composition solution, and culturing the plant tissue in the culture medium.

18. A sterilizing agent for a culture medium for culturing a plant tissue, being composed of a powdery agent containing at least sucrose fatty acid esters and nisin.

19. The sterilizing agent according to claim 18, wherein the powdery agent is composed of a plurality of powdery agents mixed together.

20. The sterilizing agent according to claim 18, further containing oxolinic acid.

21. The sterilizing agent according to claim 20, further containing natamycin.

22. The sterilizing agent according to claim 21, being prepared so that, when the sterilizing agent is added to a culture medium, the culture medium will contain 0.1-10 mg/L of nisin A, 0.1-100 mg/L of oxolinic acid, and 1-1000 mg/L of natamycin.

23. The sterilizing agent according to claim 18, further containing at least one substance selected from a group of fatty esters, salmine, egg-white lysozyme, and captan.

24. A culture medium composition for culturing a plant tissue, containing a sterilizing agent and a powdery culture-medium component, the sterilizing agent composed of a powdery agent containing at least sucrose fatty acid esters and nisin.

25. The method for preparing a culture medium for culturing a plant tissue according to claim 15, wherein the agent composing the second sterilizing agent contains one or more substances selected from a group of captan, oxolinic acid, and hypochlorite.

26. The sterilizing agent according to claim 20, further containing at least one substance selected from a group of fatty esters, salmine, egg-white lysozyme, and captan.

27. The sterilizing agent according to claim 21, further containing at least one substance selected from a group of fatty esters, salmine, egg-white lysozyme, and captan.

28. The sterilizing agent according to claim 22, further containing at least one substance selected from a group of fatty esters, salmine, egg-white lysozyme, and captan.

29. The culture medium composition for culturing a plant tissue according to claim 24, wherein the sterilizing agent contains oxolinic acid.

30. The culture medium composition for culturing a plant tissue according to claim 29, wherein the sterilizing agent further contains namatycin.

31. The culture medium composition for culturing a plant tissue according to claim 24, wherein the sterilizing agent further contains at least one substance selected from a group of fatty esters, salmine, egg-white lysozyme, and captan.
